(19) United States
(12) Patent Application Publication
(10) Pub. No.: US 2007/0083087 A1
(43) Pub. Date: Apr. 12, 2007

(54) Fixator with membrane

(75) Inventor: Daniel D. Carda, Piedmont, SD (US)

Correspondence Address: REINHART BOERNER VAN DEUREN P.C., 2215 PERRYGREEN WAY, ROCKFORD, IL, 61107, US

(73) Assignee: Sismed, LLC, Rapid City, SD (US)

(21) Appl. No.: 11/248,627

(22) Filed: Oct. 12, 2005

(51) Int. Cl. A61B 1/32

(52) U.S. Cl. 600/236

(57) ABSTRACT

A surgical instrument releasably securable to an eye using suction is provided. The surgical instrument comprises a body, a handle, and a porous member. The body forms an annular suction cavity. The annular suction cavity receives the suction and has an open end in a concave lower surface. The open end is directed toward the eye. The handle is integrally formed with the body. The handle extends upwardly away from the eye and radially outwardly from the body. The handle includes a passage adapted to deliver the suction to the suction cavity. The porous member is disposed within the suction cavity. The porous member is adapted to restrict absorption of the eye into the suction cavity through the open end and to permit the suction to permeate the suction cavity and reach the open end such that the surgical instrument is releasably securable to the eye using the suction.

### FIELD OF THE INVENTION

This invention relates generally to surgical instruments and, in particular, to a surgical instrument for use in the field of ophthalmology.

### BACKGROUND OF THE INVENTION

Eye surgeons (i.e., ophthalmologists) are able to treat maladies of the eye as well as correct certain vision problems by performing a host of different surgical procedures. For example, a surgeon can perform an LRI (limbal relaxing incisions) procedure to correct astigmatism. Astigmatism in the eye is the result of two mutually perpendicular meridians of the anterior face of the cornea failing to possess the same curvature. The LRI procedure often comprises forming arcuate incisions in the cornea, with a trephine or scalpel, in an attempt to relax or reshape the cornea to a more spherical shape and/or more closely match the perpendicular meridians. The incisions are generally made perpendicular to the most highly curved meridian (i.e., the meridian with the shorter radius of curvature) and disposed on opposite sides of the cornea.

Another common procedure performed by the eye surgeon is cataract surgery to remove a cataract. A cataract is any opacity that has developed in the crystalline lens of the eye or envelope. Cataracts can be partial or complete, progressive or stationary, and hard or soft. Cataract surgery, which is the most effective and common treatment for cataracts, involves the eye surgeon removing or repairing the cloudy or otherwise damaged lens. To do so, the eye surgeon makes an incision in the cornea of the eye to create an opening that exposes the damaged lens. Using that opening, the eye surgeon implants an intraocular lens in the eye, either with or without removing the damaged natural lens, such that vision is improved or restored.

In addition to the above LRI and cataract procedures, eye surgeons are further called upon to perform a penetrating keratoplasty (PK) procedure, which is otherwise known as corneal transplant surgery or corneal graft surgery. This procedure is done to remove a cloudy and/or diseased cornea and replace it with a clear donor cornea. To complete this procedure, the eye surgeon can utilize one of a variety of different devices and employ various methods. In one instance, the eye surgeon first removes a “button” or graft of corneal tissue from a donor cornea. This donor button can be formed and removed using one of many surgical instruments such as, for example, a “punch”, a drill, a trephine, a scalpel, or a scissors.

Undesirably, the punch for the eye surgery is much like the punch used to make an adjustment hole in a belt. When such a punch is used, the corneal tissue at the periphery of the cornea and proximate the top and bottom surfaces bunches and/or becomes distorted as the cornea is compressed. Resultantly, the peripheral wall facing radially outwardly and progressing circumferentially around the donor button lacks uniformity, is not planar, is not smooth, and the like.

In lieu of the punch method of forming the donor button, quite often a trephine or scalpel is used to begin forming the donor button. After a good portion of the donor button has been formed by trephination or using the scalpel, the scissors is used to separate the rest of the button from the remainder of the donor cornea. Resultantly, these buttons all too often have irregular sizes and shapes as well as and jagged edges. Notably, neither the punch method or trephine/scalpel/scissors methods are particularly precise.

Despite which method is chosen to form the donor button, thereafter the eye surgeon turns his attention to the eye of the patient (or recipient) of the donor tissue. First, the eye surgeon uses the trephine or a scalpel to remove a damaged portion of the cornea from the eye of the patient. The removal of the damaged portion forms a “bed” in a central portion of the cornea. If the surgeon is extremely skillful, and with any luck, the bed and the previously formed button are very similarly sized and dimensioned. Unfortunately, this is not often the case. Nonetheless, the donor button is maneuvered into the bed by the eye surgeon, the donor button is secured to the eye of the patient with a stitch or stitches, other surgical procedures are performed, and the patient is permitted to heal. Thereafter, depending on how closely matched in size, shape, and dimension the button and the bed were to each other, the vision of the patient is restored or improved to some degree. However, if the button and bed were not closely matched, the result is often a moderate to severe astigmatism.

As each of the above-described surgeries illustrate, the eye surgeon is often tasked with making one or more extremely precise incisions in the eye. These incisions are often millimeters in length or, in some procedures, mere fractions of a millimeter. To further complicate matters, in many procedures these small incisions must also be accurately located on the eye based on, for example, nomograph data and information. If either or both of the size and position of an incision is inaccurate, the surgical procedure might well yield less than favorable results. Resultantly, the eye surgeon must ensure that the eye is stabilized and fixed when making these delicate incisions. To that end, the eye surgeon typically relies upon a surgical instrument known as a fixator (e.g., a globe fixator). The fixator is a devices that is releasably secured to the cornea of the eye such that relative positioning of another surgical device (e.g., the trephine or the scalpel) is aided or guaranteed by not allowing the patient to move his or her eye during such procedure.

In order to be releasably secured to the eye, several of the fixators known in the art employ teeth, hooks, barbs, and/or suction (or a suction force). When suction is used, the suction is typically created by a peristaltic pump or a spring-loaded syringe. The suction is used to generate a vacuum (or partial vacuum) in a suction cavity within the fixator. Since the suction cavity has an open end generally oriented and directed downwardly, when the fixator is lowered upon the anesthetized eye of a patient, the vacuum within the suction cavity clamps the fixator to the eye and draws the two together. As such, the eye and the fixator are releasably secured to each other, relative movement between the two is restricted or altogether prohibited, and the eye surgeon is able to use the fixator to position other surgical instruments proximate the eye as needed.

Unfortunately, when suction is used to create the vacuum in the suction cavity and releasably secure the fixator to the eye, one or more portions of the corneal tissue are drawn upwardly and/or pulled into the suction cavity. As this happens, the corneal tissue may become unnaturally distorted and blood vessels in the eye may be damaged. Furthermore, the suction cavity or suction passage can becomes occluded by the corneal tissue such that an uneven distribution of suction results.

Therefore, a fixator that can be releasably secured to the eye using suction, without causing the aforementioned difficulties, would be desirable. The present invention provides such a fixator and is directed to overcoming one or more of the problems as set forth above. Advantages of the invention, as well as additional inventive features, will be apparent from the description of the invention provided herein.

Also, the trephines known in the art are unsatisfactory for creating perfectly arcuate incisions at one or more precise locations. Therefore, surgeries such as the LRI procedure are not as effective as they could be. Moreover, methods and devices used to create the donor button and the bed in the cornea of a patient during the PK procedure result in buttons that are jagged, not circular, mismatched, and the like.

Therefore, a trephine or trephine system that can alleviate these disadvantages would be desirable. The present invention provides such a trephine and trephine system and is directed to overcoming one or more of the problems as set forth above. Advantages of the invention, as well as additional inventive features, will be apparent from the description of the invention provided herein.

### BRIEF SUMMARY OF THE INVENTION

In view of the above, the present invention provided a new and improved fixator. More particularly, the present invention provided a new and improved fixator for use in performing eye surgeries such as, for example, an LRI procedure, cataract surgery, and a PK procedure.

In one embodiment of the present invention, the fixator may be advantageously secured to the cornea of the eye without drawing, pulling, or absorbing corneal tissue into the suction cavity. As such, the corneal tissue is prevented from becoming unnaturally and grossly distorted when the fixator is in intimate contact with the eye. Also, by not drawing corneal tissue into the suction cavity, the potential for damaging blood vessels in the eye is reduced and the suction cavity or suction passage is protected from becoming occluded by the corneal tissue.

In one aspect, the invention provides a surgical instrument releasably securable to an eye using suction. The surgical instrument comprises a body and a porous member. The body forms a suction cavity having an open end. The porous member is disposed within the suction cavity. The porous member is adapted to restrict absorption of the eye into the suction cavity through the open end and to permit the suction to permeate the suction cavity and reach the open end such that the surgical instrument is releasably securable to the eye using the suction.

In another aspect, the invention provides a surgical instrument releasably securable to an eye using suction. The surgical instrument comprises a body and a porous member. The body forms an annular suction cavity. The annular suction cavity receives the suction and has an open end in a concave lower surface. The open end is directed toward the eye. The porous member is disposed within the suction cavity. The porous member is adapted to restrict absorption of the eye into the suction cavity through the open end and to permit the suction to permeate the suction cavity and reach the open end such that the surgical instrument is releasably securable to the eye using the suction.

In yet another aspect, the invention provides a surgical instrument releasably securable to an eye using suction. The surgical instrument comprises a body, a handle, and a porous member. The body forms an annular suction cavity. The annular suction cavity receives the suction and has an open end in a concave lower surface. The open end is directed toward the eye. The handle is integrally formed with the body. The handle extends upwardly away from the eye and radially outwardly from the body. The handle includes a passage adapted to deliver the suction to the suction cavity. The porous member is disposed within the suction cavity. The porous member is adapted to restrict absorption of the eye into the suction cavity through the open end and to permit the suction to permeate the suction cavity and reach the open end such that the surgical instrument is releasably securable to the eye using the suction.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1 is a perspective view of an exemplary embodiment of a fixator constructed in accordance with the teachings of the present invention;

FIG. 2 is a cross section view of the fixator of FIG. 1 disposed upon an eye;

FIG. 3 is a perspective view of an adjustable trephine adapted for use with the fixator of FIG. 1;

FIG. 4 is a perspective view of an alternate embodiment an adjustable trephine adapted for use with the fixator of FIG. 1;

FIG. 5 is a partial cross section view of the fixator of FIG. 1 engaged with the adjustable trephine of FIG. 3 and disposed upon the eye such that a blade from the trephine is inserted a maximum depth into a cornea of the eye;

FIG. 6 is a partial cross section view of the fixator of FIG. 1 engaged with the adjustable trephine of FIG. 3 and disposed upon the eye such that the blade from the trephine is inserted somewhat less than the maximum depth into the cornea of the eye;

FIG. 7 is a top plan view of the trephine of FIG. 3 highlighting cross hairs disposed within the trephine;

FIG. 8 is a perspective view of an alternate exemplary embodiment of a fixator constructed in accordance with the teachings of the present invention;

FIG. 9 is a perspective view of an alternate exemplary embodiment of a fixator constructed in accordance with the teachings of the present invention;

FIG. 10 is a part cross section and part elevation view of donor module from a penetrating keratoplasty unit adapted to form a donor button using a trephine; and

FIG. 11 is a part cross section and part elevation view of patient module from the penetrating keratoplasty unit adapted to form a patient button using the same trephine as illustrated in FIG. 10.

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

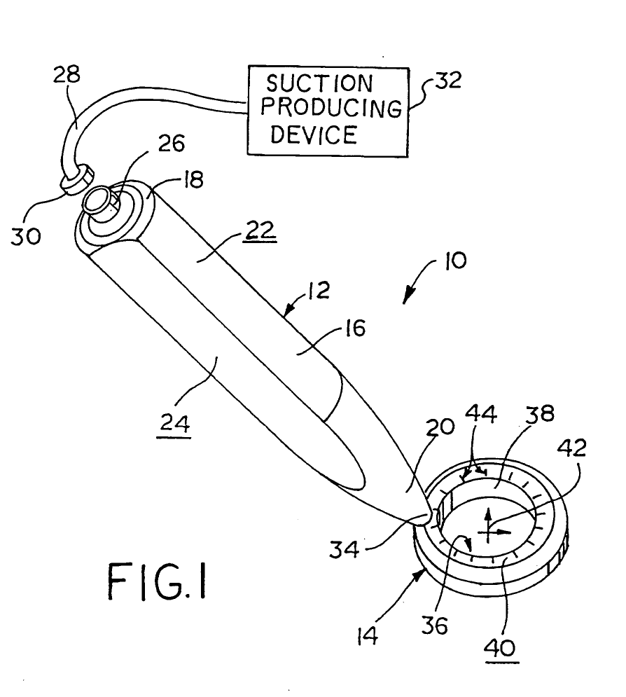

Referring to FIG. 1, a fixator 10 constructed in accordance with one embodiment of the present invention that is adapted to be releasably secured to the cornea of an eye is illustrated. The fixator 10 is preferably constructed of a rigid polymer material such as, for example, a plastic. In such an embodiment wherein the fixator 10 is formed from inexpensive and readily available materials, the fixator 10 is completely disposable. The disposable nature of the fixator 10 guarantees that a sterile device is used for each new patient. This ensures that diseases, such as bovine spongiform encephalopathy (BSE), and the like, are not transmitted and/or are prevented. In other embodiments, other materials may be used to construct the fixator 10.

As depicted in FIG. 1, the fixator 10 comprises a handle 12 and a body 14. The handle 12 is a generally cylindrical member extending upwardly and radially outward from the body 14. The handle 12 is preferably angled from the body 14 by between about 20 to about 45 degrees. In a preferred embodiment, the handle 12 is slanted about thirty degrees from vertical as shown in FIG. 1. The handle 12 defines a central portion 16 between an upper end 18 and a lower end 20. To help the eye surgeon grasp and control the fixator 10, an exterior surface 22 of the handle 12 can be outfitted with a pattern of knurls, a series of depressions, and the like. In the illustrated embodiment, the exterior surface 22 includes a planar surface 24 on each opposing side of the handle 12. These planar surfaces 22 also enable the eye surgeon to securely grip and hold the fixator 10. In one embodiment, the handle 12 is ergonomically fashioned to conform to a hand and fingers of the eye surgeon.

Proximate the upper end 18, the handle 12 is preferably equipped with a quick connect mechanism 26, assembly, or fitting. The quick connect mechanism 26 is configured to quickly and easily mate with a hose or tube 28 having a mating quick connect mechanism 30, assembly, or mating fitting. The tube 28 is coupled to a suction producing device 32 such as, for example, a pump or a spring-loaded syringe. Such a coupling provides a suction that is transmitted from the tube 28 to the handle 12 of the fixator 10. In other words, a vacuum (or more correctly a partial vacuum) is able to be introduced into the fixator 10.

The lower end 20 of the handle 12 may be generally tapered as the handle progresses closer to the body 14 in one embodiment. Where the lower end 20 of the handle 12 and the body 14 intersect, a neck 34 that couples the handle 12 and the body 14 is formed. In a preferred embodiment, the handle 12 and the body 14 are integrally formed and, as such, the neck 34 flows smoothly into the body. In other embodiments the handle 12 and the body 14 are separate components that are secured together.

As may be seen from this FIG. 1, the body 14 of this embodiment of the fixator 10 is annular or “ring like” in shape and, therefore, includes a central aperture 36, an inner wall 38, and an upper surface 40. As depicted, the central aperture 38 defines a vertical central axis 42 inside the inner wall 38 of the body 14. In the embodiment illustrated in FIG. 1, the central aperture 36 is round and the inner wall 38 is smooth and parallel with the vertical central axis 42. In other embodiments, the body may be oval, elliptical, or otherwise configured to mate with the eye.

In a preferred embodiment, the upper surface 40 of the body 14 includes one or more indicia 44 or markings. These indicia 44 can take a number of forms such as, for example, simple lines placed upon or etched into the upper surface. In more elaborate cases, the indicia 44 can be markings of a particular degree such as, for example, thirty-six lines in spaced relation about the circumference of the upper surface 40, each mark being ten degrees apart from the next such that a full circle of three hundred sixty degrees is identified for the eye surgeon. The indicia 44 can also indicate a particular axis or quadrant, employ various symbols, be distinguished by color, and the like.

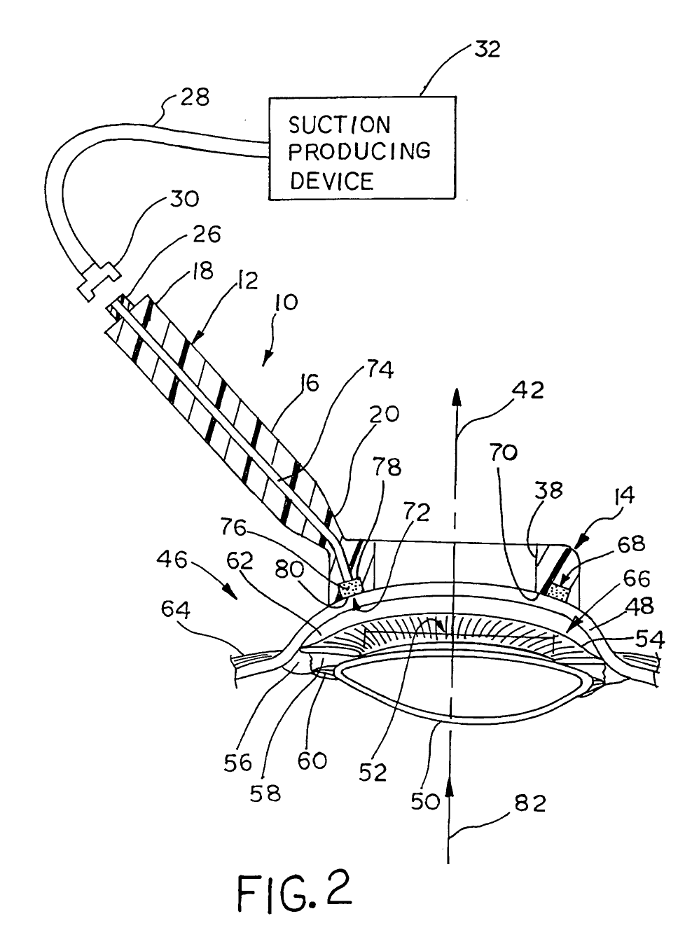

As illustrated in FIG. 2, the fixator 10 is shown disposed upon an eye 46. The partial illustration of the eye 46 reveals, among other things, a cornea, a lens 50, a pupil 52 defined by an iris 52, ciliary muscle 56 adjacent to a posterior chamber 58, suspensory ligaments 60, an anterior chamber 62, and a conjunctiva 64. The portion of the eye 46 radially outward of pupil 52, which is shown in a dilated state, is sometimes referred to as a vascular zone 66 or vascular tunic due to the prevalence of blood vessels in that area. The body 14 of the fixator 10 is sized and dimensioned such that the inner wall 38 is generally inside the vascular zone 66 of the eye 46. In other words, an inner diameter of the central aperture 36 is preferably less than a diameter of the vascular zone 66 and/or the dilated pupil 52.

The body 14 further includes a suction cavity 68 formed in the lower surface 70 of the body. The suction cavity 68 is generally a groove or an annular chamber extending preferably around the circumference of the body 14. The suction cavity 68 has an open end 72 directed toward the eye 46 when the fixator 10 is disposed or seated upon the cornea 48 of the eye 46 as shown in FIG. 2. In a preferred embodiment, the lower surface 70 of the body 14 is curved or otherwise contoured to match the curvature of the cornea 48 of the eye 46. In an exemplary embodiment, the suction cavity 68 has a depth of about one millimeter into the body 14.

The suction cavity 68 is provided with suction courtesy of a passage 74 generally between the upper and lower ends 18, 20 of the handle 12 and passing into the body 14. The passage 74 provides fluid communication from the tube 28 to the suction cavity 68 in the body 14. Therefore, a negative pressure such as a vacuum (or more accurately a partial vacuum) generated and produced by the suction producing device is carried through the handle 12, through the body 14, and into the suction cavity 68.

Still referring to FIG. 2, the suction cavity 68 is adapted to house a porous member 76. The porous member 76 permits permeation of a fluid (e.g., air) therethrough. As such, the porous member 76 permits the suction received by the suction cavity 68 to pass from a first or upper side 78 of the porous member to a second or lower side 80. Additionally, the suction is able to permeate through the porous member 76 so that the suction is evenly distributed throughout the suction cavity 68 and, in particular, uniformly dispersed proximate the open end 72.

The porous member 76 can be formed from a natural material, a synthetic material, or some combination thereof. The porous member 76 can be a single piece of material or several pieces of material adjacent to each other, bonded together, and the like. The porous member 76 can be held within the suction cavity 68 by a slight friction fit and/or by other methods or means for securement such as, for example, a small amount of epoxy. In an exemplary embodiment, the porous member 76 has a thickness of about one millimeter to correspond to the depth of the suction cavity 68. In one embodiment, the porous member 76 is a porous membrane such as, for example, a microporous membrane that permits passage of micron sized particles while restricting the penetration of larger particles.

In operation, the fixator 10 of FIGS. 1 and 2, is used by the eye surgeon both or either as a guide or reference device during a surgical procedure such as, for example, the LRI procedure to correct an astigmatism, and/or to hold the eye 46 of the patient steady during the procedure. During this procedure, the eye surgeon first prepares the eye 46 of the patient by anesthetizing the eye. Thereafter, the eye surgeon may stamp or otherwise mark the eye 46 with ink to indicate a visual axis 82 of the eye, a location or locations where one or more incisions are needed, quadrants or meridians of the eye, and the like. Such markings are typically based upon data and information obtained from a nomograph or eye tests and/or the knowledge and experience of the eye surgeon.

With the eye 46 of the patient prepared, the fixator 10 is lowered onto the eye as generally shown in FIG. 2. In a preferred embodiment, the fixator 10 is lowered upon the eye 46 such that the visual axis 82 of the eye is aligned with the vertical central axis 42 of central aperture 36. In other circumstances, the fixator 10 can be placed upon the eye 46 by aligning the fixator with the cornea 46 or by using other landmarks of the eye or surrounding structure as a reference as known in the art.

After the fixator 10 is desirably located, the suction producing device 32 is activated such that suction is generated and then conducted through the tube 28 and the passage 74 until the suction reaches the suction cavity 68. Once the suction has reached the suction cavity 68, the suction permeates through the porous member 76 and passes through to the open end 72 where the suction is preferably uniformly circumferentially distributed. The suction at the open end 72 causes the fixator 10 to clamp down upon the eye 46 such that the fixator and the eye are releasably secured together. In this state, relative movement between the fixator 10 and the eye 46 is inhibited, restricted, and preferably prevented altogether.

Advantageously, when the fixator 10 and eye 46 are held together by the suction, the porous member 76 inhibits or prevents the cornea of the eye (or corneal tissue) from being drawn into the suction cavity 68. As a result, the corneal tissue is not unnaturally distorted and the potential for damaging blood vessels in the eye 46 is reduced. Furthermore, the suction cavity 68 is protected from becoming occluded by the corneal tissue.

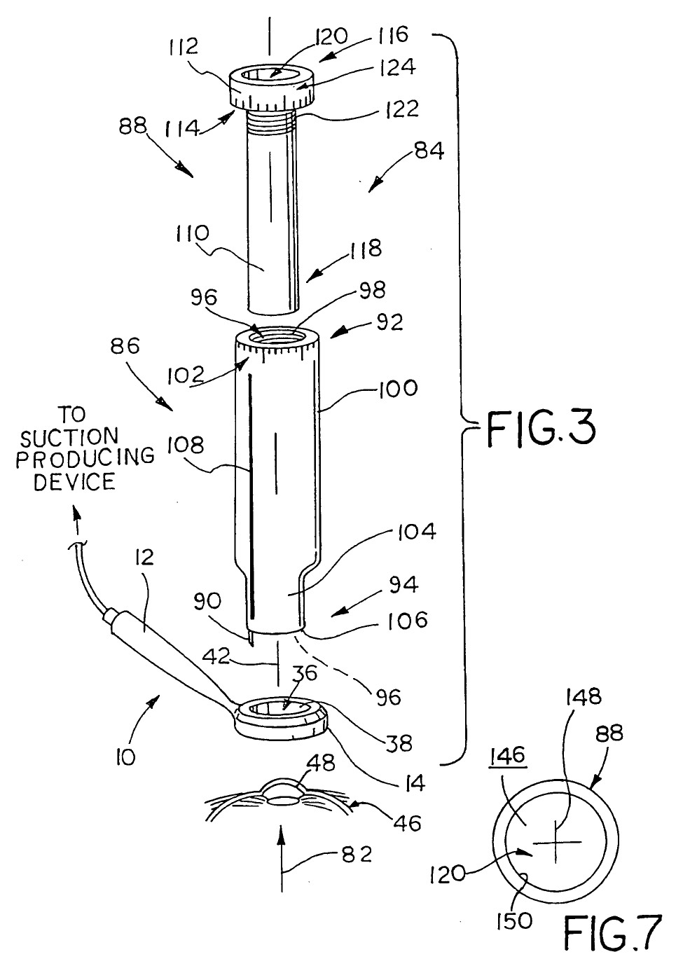

With the eye 46 comfortably and releasably clamped to the fixator 10, the eye surgeon next employs a trephine 84, as illustrated in FIG. 3, to make the delicate incisions. The trephine 84 comprises a blade carrier 86, an applanator 88, and a blade 90. The blade carrier 86 is a generally hollow cylindrical tube or body that has a glare-free outer surface (e.g., a satin surface), defines an upper end 92 and a lower end 94, and has a central bore 96 extending between the upper and lower ends. In one embodiment, threads 98 are formed on an inner wall of the central bore 96, preferably proximate the upper end 92 of the blade carrier 86, although such threads maybe formed proximate the lower end 94 or otherwise within the central bore 96. In an exemplary embodiment, a diameter of the inner wall where the threads 98 are located is about three hundred seventy-five thousandths of an inch in diameter and the threads are dimensioned such that one tenth of a millimeter of axial travel (i.e., vertical movement) will occur per revolution of the applanator 88 relative to the blade carrier 86. Preferably, the diameter of the inner wall is between about three tenths of an inch and half an inch and the axial travel per revolution is less than a millimeter.

In a preferred embodiment of the present invention, the upper end 92 of the body 100 of the blade carrier 86 may also include a plurality of indicia 102 circumferentially spaced on an external surface. The indicia 100 or markings are preferably lines, numbers, and/or symbols separated by a known distance such as, for example, by a millimeter. As such, a scale is provided at the upper end 92 of the body 100 for use by the eye surgeon in one embodiment.

As the body 100 of the blade carrier 86 progresses toward the lower end 94, the body 100 tapers to form a fixator engagement portion 104. The fixator engagement portion 104 is sized and dimensioned to be telescopically and rotatably received within the central aperture 36 of the fixator 10. Preferably, there is little tolerance between the inner wall 38 and the fixator engagement portion 104 when the two are coupled. In other words, the fixator engagement portion 104 is snugly fit within the central aperture 36 while still permitting rotation. In an exemplary embodiment, the diameter of each piece should not vary by more than about one thousandths of an inch, and the roundness must be maintained to a similar level.

The blade 90 is secured to the lower end 94 of the body 14 as shown in FIG. 3. The blade 90 is mounted such that it is parallel with the body 100 of the blade carrier 86 as well as the inner wall 38 and/or vertical central axis 42 of the fixator 10 when the fixator and the blade carrier 86 are coaxially engaged. For the purposes of illustration, the size of the blade 90 has been exaggerated. In a preferred embodiment, the blade 94 projects only a few tenths of a millimeter (e.g., seven tenths of a millimeter) from a distal edge 106 at the lower end 94 of the blade carrier 86. While a variety of different blade lengths are employable, preferably the blade length is between about seven tenths of a millimeter to about five tenths of a millimeter.

To mark the position of the blade 90, an indicium 108 or marking is placed upon or formed in the external surface of the body 100 of the blade carrier 86. The indicium 108 is in vertical alignment with the blade 90 as shown in FIG. 3 and, therefore, when using the trephine 84 during a surgical procedure, the eye surgeon is notified and advised as to the exact position of the blade. This is true even when the blade 90 and the fixator engagement portion 104 of the blade carrier 86 are hidden from view by the fixator 10.

Still referring to FIG. 3, the applanator 88 is a generally cylindrical tube or body 110 integrally formed with an axially-aligned, annular top 112. In one embodiment, the cylindrical tube or body 110 may be hollow. The annular top 112 preferably has a diameter that is greater than the diameter of the body 110. As such, the annular top 112 forms a shoulder 114 or abutting surface that intersects with or engages the body 110. The applanator 88 defines an upper end 116 proximate the annular top 112 and a lower end 118 in spaced relation to the upper end as illustrated in FIG. 3. A central bore 120 passes through the annular top 112 and the body 110 and generally extends between the upper and lower ends 116, 118 of the applanator 88.

In an embodiment adapted to mate with an embodiment of the blade carrier having threads proximate an upper end thereof, just below the annular top 112 and proximate the shoulder 114, an exterior surface of the body 110 proximate the upper end 116 includes threads 122. These threads 122 are sized and dimensioned to mate with the threads 98 inside the central bore 96 of the blade carrier 86. Therefore, in an exemplary embodiment, a diameter of an exterior surface where the threads 122 are located is about three hundred seventy-five thousandths of an inch in diameter and the threads are dimensioned such that one tenth of a millimeter of axial travel (i.e., vertical movement) will occur per revolution of the applanator 88 relative to the blade carrier 86. Preferably, the diameter of the inner wall is between about three tenths of an inch and half an inch and the axial travel per revolution is less than a millimeter.

The annular top 112 of the applanator 88 also includes a plurality of indicia 124 circumferentially spaced on the external surface. The indicia 124 or markings are preferably lines, numbers, and/or symbols separated by a known distance such as, for example, by a millimeter. As such, a scale is provided at the upper end 116 of the body 110. The indicia 124 of the annular top 112 are preferably configured to correspond to the indicia 102 on the body 100 of the blade carrier 86.

Still referring to FIG. 3, when the applanator 88 is lowered, the body 100 of the applanator is telescopically received in the central bore 96 of the blade carrier 86 until the threads 122, 98 are encountered. Thereafter, by rotating the applanator 88 relative to the blade carrier 86, the two components become threadably engaged. In such a threaded engagement, the applanator 88 can be threadably rotated relative to the blade carrier 86 such that the applanator is driven either axially upwardly or axially downwardly with respect to the blade carrier. In one embodiment, the pair of threads 98, 122 include a cooperating detent and follower, as well known in the art, such that discrete positions can be found, felt, and/or heard when rotating the threadably engaged components.

Notably, the indicia 102, 124 on the blade carrier 86 and the applanator 88 can be used by the eye surgeon as a guide or reference tool to correlate relative rotational movement to relative axial movement. For example, if the eye surgeon needs to move the applanator 88 three tenths of a millimeter further into the blade carrier 86, the eye surgeon simply turns the applanator clockwise until three of the indicia 124 pass a fixed one of the indicia 102 on the blade carrier 86. In contrast, if the eye surgeon wants to move the applanator 88 five tenths of a millimeter out of the blade carrier 86, the eye surgeon rotates the applanator counterclockwise until five of the indicia 124 have passed a fixed one of the indicia 102 on the blade carrier. As will become apparent when more fully discussed below, this relative axial movement permits the blade 90 to be adjusted to an infinite number of different depths. Also, once the applanator 88 and blade carrier 86 have been rotated into a desired position using the indicia 124, 102 as a guide, relative axial movement between the applanator and blade carrier is prohibited by the engagement of the threads 98, 122. In other words, once the desired position is achieved, the applanator 88 and the blade carrier 90 are “locked” into that position.

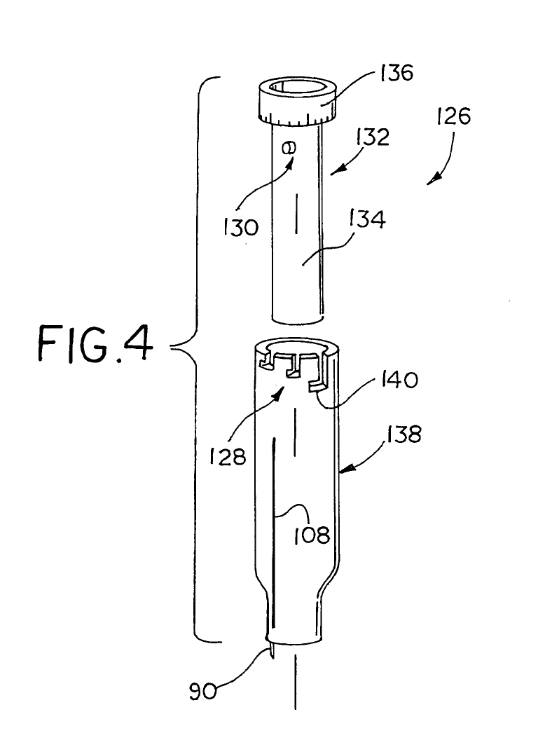

In an alternate embodiment as shown in FIG. 4, the trephine 126 includes cooperating slots 128 and a stud 130 to set the depth of the blade relative to the applanator 132. In the illustrated embodiment, the applanator 132 is outfitted with the stud 130 on the outer surface of the body 134 near the annular top 136 and the blade carrier 138 is fashioned with the slots 128. When the applanator 132 is telescopically engaged with the blade carrier 138, the stud 130 and a selected one of the slots 128 are engaged. The stud 130 is “locked” within the selected one of the slots 128 by rotating the applanator 132 with respect to the blade carrier 138 to place the stud in the horizontal portion 140 of the chosen slot. Since each of the slots 128 have a different depth (e.g., sixty, sixty-five, and seventy hundredths of a millimeter), the blade 90 on the blade carrier 138 is adjustable similarly to the trephine 84 (FIG. 3) using mating threads 98, 122. As those skilled in the art will recognize, alternate methods and systems for axially adjusting the applanator 88, 132 relative to the blade carrier 86, 138 to control blade depth and for “locking” the applanator and blade carrier together to prevent relative axial movement can be employed. Preferably, the blade depth can be adjusted to, and locked, at of depth of about seven tenths of a millimeter to less than about five tenths of a millimeter or less.

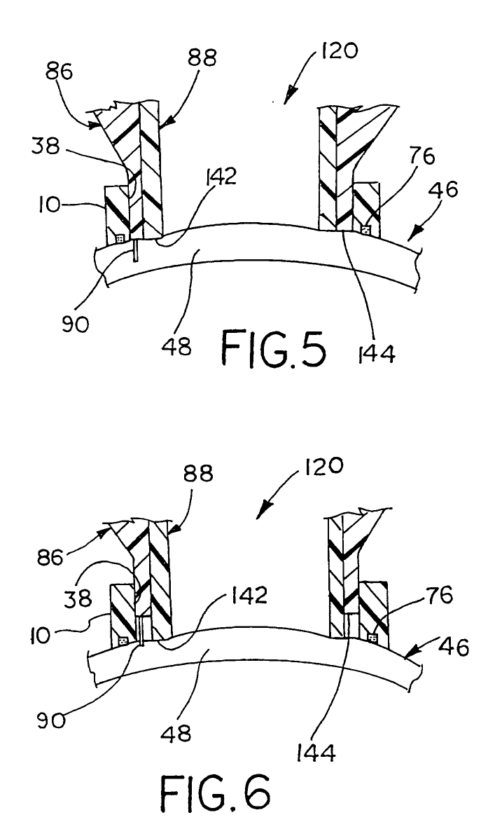

Referring back to FIG. 3, once the fixator 10 is secured to the eye as previously discussed and the blade 90 of the trephine 84 has been adjusted by rotating the applanator 88 and the blade carrier 86 relative to each other, the assembled and axially “locked” trephine 84 is lowered into, and telescopically received within, the central aperture 36 of the fixator. When this occurs, as best shown in FIGS. 5 and 6, an applanating surface 142 on the applanator 88 slightly flattens the cornea 48 of the eye 46. Simultaneously, the blade 90 on the blade carrier 86 penetrates, but does not pierce, the cornea 48. Since the blade 90 is parallel to the inner wall 38 of the fixator 10, the blade is inserted straight down into the cornea 48. If an angled or curved blade is employed on the trephine, care should be used to ensure that the blade enters the cornea generally transverse to the surface of the cornea to prevent damaging the cornea.

As illustrated in FIG. 5, the blade 90 is depicted in a position where the blade has entered the cornea 48 to a maximum depth (e.g., about seven tenths of a millimeter). In such a circumstance, the applanating surface 142 is generally planar with a distal end 144 of the blade carrier 86 and, the applanator 88 has been threadably driven down until the shoulder 114 (FIG. 3) abuts and engages the upper end 92 of the blade carrier 86.

However, if the eye surgeon had desired to insert the blade 90 into the cornea 48 to a smaller depth (e.g., about six tenths of a millimeter), prior to insertion of the trephine 84 into the fixator 10 the surgeon would have axially adjusted the applanator 88 with respect to the blade carrier 86 such that the applanating surface 142 projects past the distal end 144 as shown in FIG. 6. In other words, by rotating the applanator 88 relative to the blade carrier 86, the depth of the blade 90 can be adjusted. As FIG. 6 shows, the blade 90 has been raised and penetrates the cornea 48 less than the blade penetrated the cornea in FIG. 5.

As described above, the depth of the blade 90 can be determined and/or calculated using the indicia 124, 102. First, the starting, initial, or reference position of the blade 90 is determined by threadably driving the applanator 88 down until the shoulder 114 (FIG. 3) abuts and engages the upper end 92 of the blade carrier 86, until the applanating surface 142 and the distal end 144 of the blade carrier 86 are co-planar, or some other method of establishing a reference point is performed. After the initial reference position is determined, adjustment of the blade is preferably performed using a threaded engagement. As such, the depth of the blade 90 can be set to an infinite number of settings by threadably drawing the blade 90 upwardly relative to the applanator 88. Thereafter, the blade 90 can be axially locked into position. The blade 90 can be locked using the engaging slots (FIG. 4), which restrict axial movement, or the blade can be locked by employing threads with a very close tolerance and sufficient frictional resistance between threaded components to resist rotation when the trephine is used to make incisions. Since the blade can be manipulated to a desired position and locked in place, the depth of the blade 90 can be customized or tailored for each individual patient.

To customize or tailor the blade depth for a particular patient, the eye surgeon first measures the corneal thickness with a pachymeter. Thereafter, a corneal topographer is employed to measure the amount of astigmatism in the eye and to determine the needed length of incision to correct that astigmatism. The information obtained from the pachymeter and the corneal topographer is then entered into the nomogram in order to calculate the optimal depth and length of incision for that patient.

With the trephine 84 positioned within the fixator 10, the eye surgeon is able to rotate the entire trephine to make an arcuate incision in the cornea 48 of the eye 46. Since the inner wall 38 of the fixator 10 is preferably circular, the incisions made in the cornea 48 by the rotating trephine 84 are also arcuate. Stated another way, the fixator 10 basically provides the trephine 84 with a guided path during rotation. As well known to those skilled in the art, precise arcuate incisions permit astigmatism in the eye 46 to be more accurately treated. In contrast, incisions that are jagged, uneven, improperly located, and the like, tend to produce somewhat poor and unexpected results after the LRI procedure has been performed.

In a preferred embodiment, the trephine 84 is sized and dimensioned such that the blade 90 creates arcuate incisions with a radius of about five and one-half millimeters. Using such a radius ensures that the relaxing incision or incisions are interior to the vascular zone 66 and the dilated pupil 52 of the eye 46 (FIG. 2). Further, in order to ensure that the appropriate length of arcuate incision is made in the cornea 48, the eye surgeon can reference the blade indicium 108 on the body 100 of the blade carrier 86 with the indicia 44 on the upper surface 40 of the fixator 10 (FIG. 1).

Moreover, as depicted in FIG. 7, to aid the eye surgeon in making the required relaxing incisions, a transparent member 146 that includes indicia such, for example, a cross hairs 148 is disposed within the central bore 120 of the applanator 88 in a generally transverse orientation with respect to the inner wall 150. As such, when the eye surgeon peers down through the trephine 84 from above (i.e., axially) to view the eye 46 during a surgical procedure, the eye surgeon sees the cross hairs 148 as shown in FIG. 7. The eye surgeon can align these cross hairs 148 with indicia or markings that have been for example, stamped with ink on the eye as a result of the data and information obtained from the nomograph. Therefore, the trephine 84 can be oriented with respect to the eye 46.

Once the incisions have been formed in the cornea 48 of the eye 46, further care of the eye is typically performed (e.g., stitches, sutures, etc.) while the eye is held immobile. After this further care has been completed, the suction producing device 32 is deactivated. With the suction removed, the fixator 10 is released from intimate contact with the eye 46 and can be gently raised away from the eye. Thereafter, the quick connect assembly 26 on the handle 12 and the mating quick connect assembly 30 on the tube 28 can be disengaged and the fixator 10 and the tube 28 disposed of in an appropriate manner such as, for example, in a medical waste receptacle. When another procedure is to be performed on the eye of a new patient, a new sterile fixator and new sterile tube are used. To encourage the use of a new sterile fixator 10 and tube 28, the fixator and/or the tube can be provided in a sterile, single use package.

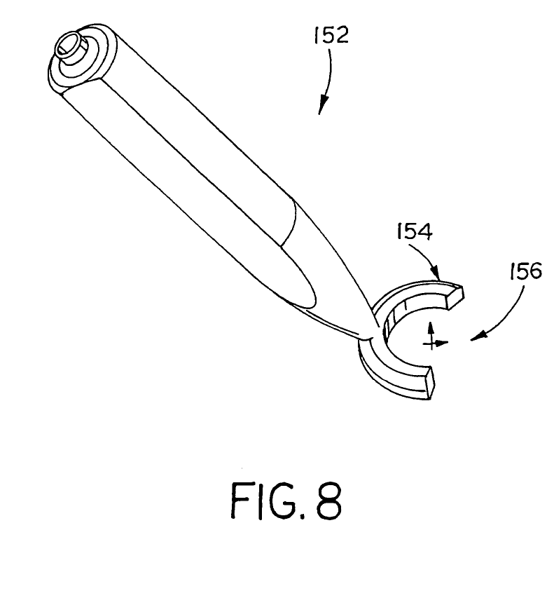

As shown in FIG. 8, an alternative embodiment of a fixator 152 is illustrated. The fixator 152, which generally includes all of the features and provides all of the advantages of fixator 10, has a semi-circular body 154 that forms and provides an open side 156. The open side 156 offers the eye surgeon room to maneuver a scalpel or other surgical instrument when, for example, cataract surgery is performed. Like above, the eye surgeon prepares the eye 46, aligns the fixator 152 and the eye, and then releasably secures the fixator to the eye using suction. With the fixator 152 and the eye 46 releasably secured together, the eye surgeon is able to make an incision in the eye proximate the open side 156 to expose the cataract in the lens of the eye. With the lens now accessible, the eye surgeon is able to repair or replace the cloudy, damaged, and/or diseased lens, perform other or further care, and then release the fixator 152 from engagement with the eye 46. As will be recognized by those skilled in the art, the open side 156 of the fixator 152 beneficially provides the eye surgeon with additional accessibility to the eye as well as additional room proximate the eye to maneuver a surgical instrument.

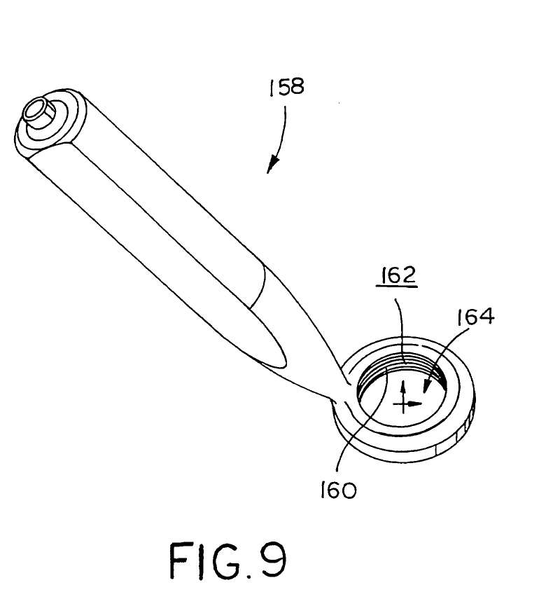

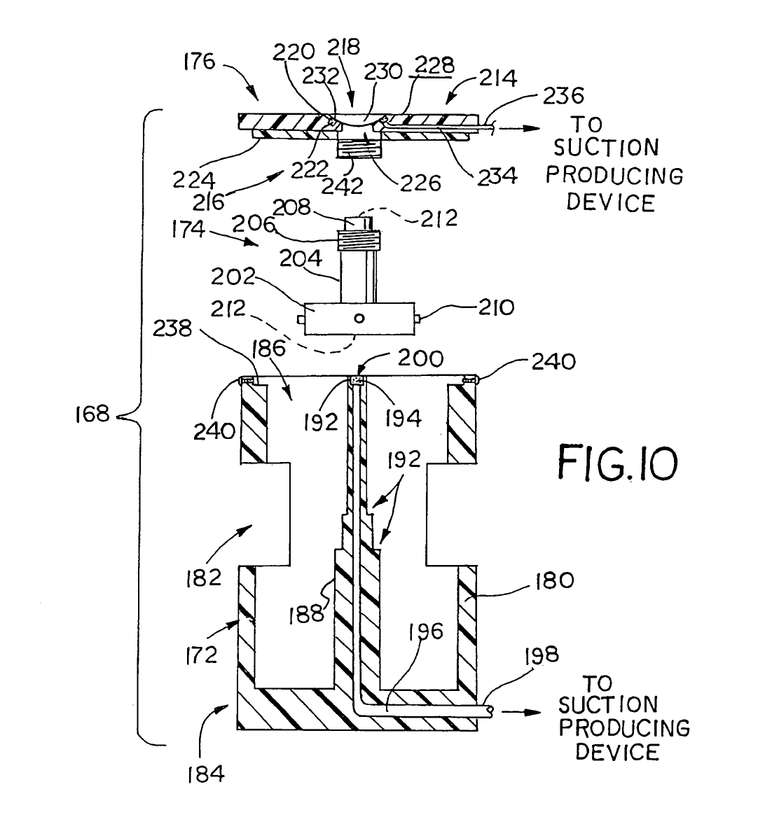

As shown in FIG. 9, yet another embodiment of a fixator 158 is illustrated. The fixator 158, which generally includes all of the features and provides all of the advantages of fixator 10, has threads 160 on an inner wall 162. The threaded inner wall 162 permits a trephine that has mating threads, as will be more fully discussed below, to be threadably engaged with the fixator 158. As such, when the fixator 158 is releasably secured to the eye, the trephine can be threadably driven downwardly into the central aperture 164 of the fixator 158 and toward the eye. As the trephine continues to be driven downwardly, eventually a blade on the trephine contacts the cornea of the eye. As the trephine continues to be rotated, perfectly arcuate incisions can be made in the cornea.

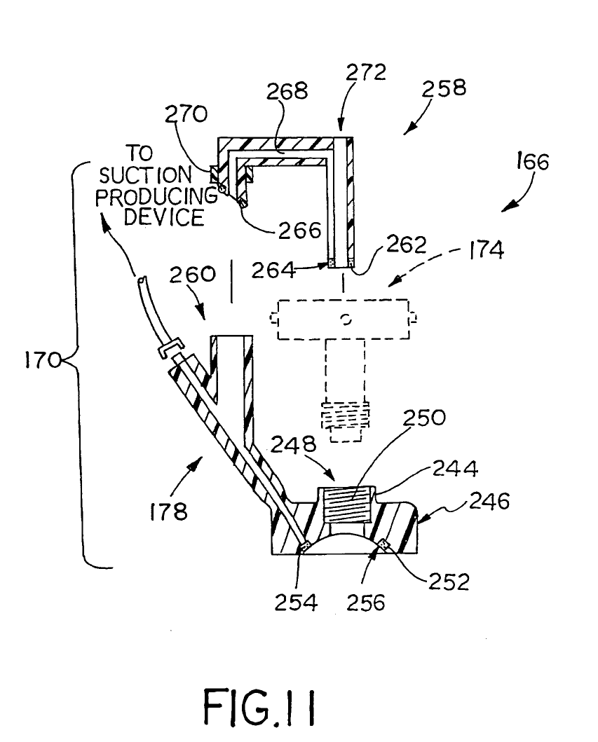

Referring now to FIG. 10, a penetrating keratoplasty unit 166 (PK unit) particularly suited for performing PK surgery is illustrated. The PK unit 166 comprises a donor module 168 and a recipient module 170. The donor module 168 includes a base 172, a trephine 174, and a cap 176. The recipient module 170 comprises a fixator 178 and the same trephine 174 (shown in dashed lines) that was included in the donor module. Notably, the trephine 144 and fixator 178 include many of the same features and provide many of the same advantages as the trephines 84, 126 and fixators 10, 152, 158 previously described.

The base 172 of the donor module 168, which is illustrated in cross section in FIG., has a generally cylindrical body 180 reduced in diameter by a centrally disposed access cut-out 182. The base 172 defines a closed lower end 184 and an open upper end 186. Projecting upwardly from the closed lower end 184, the base 172 has a central stabilizer 188. The central stabilizer 188 is generally a fixed hollow shaft that has steps 192 formed in an exterior surface by a series of decreasing shaft diameters as the shaft continues to progress toward the open upper end. Proximate the open upper end 186 of the base 172, the stabilizer 188 includes a suction cavity 192 housing a porous member 194. A passage 196 in the base 172 and stabilizer 188 extends from a suction port 198 in the base 172, through the stabilizer 188, and to the suction cavity 192. Therefore, suction introduced by the suction producing device 32 (FIG. 1) can be transmitted to the suction cavity 192. As before, the porous member 194 permits the suction in the suction cavity 192 to permeate therethrough and be evenly distributed at the open end 200 of the suction cavity.

The trephine 174 of FIG. 10 includes a thumb wheel 202, a generally cylindrical central body portion 204, threads 206, and a blade 208. The thumb wheel 202 is an annular or circular member that is designed to be gripped and rotatably acted upon by the eye surgeon. In that regard, the thumb wheel 202 may include one or more gripping members 210 such as, for example, knobs, knurls, studs, depressions, and the like. The body portion 204 is interposed between the thumb wheel 202 and the threads 206. The threads 206 are sized and dimensioned to permit about one tenth of a millimeter of axial travel per revolution of the trephine 174. The blade 208 is preferably a circular trephine blade having a central opening as well known by those skilled in the art.

The entire trephine 174 in FIG. 10 includes an axial bore 212 formed therethrough such that the trephine can be lowered down and telescopically received upon the stabilizer 188 of the base 172. In fact, stabilizer 188 is able to pass through the axial bore 212 of the trephine 174 until the trephine comes to rest on one of the steps 192. When disposed on the stabilizer 188, the thumb wheel 202 of the trephine 174 is accessible by the eye surgeon through the access cut-out 182.

The cap 176 defines an upper end 214 and a lower end 216 and includes a concave cavity 218, a suction cavity 220 housing a porous member 224, a circular flange 226 or deck, and an axial bore 228. The concave cavity 218 is formed in a top surface 228 of the cap 176 and defines a partially hemispherical or curved wall 230. The wall 230 is contoured such that a cornea 48 or an entire eye 46 (i.e., the entire globe of the eye) can be seated in the concave cavity 218. The concave cavity 218 is partially bordered by the suction cavity 220 such that an open end 232 of the suction cavity is exposed to the concave cavity. A passage 234 in the cap 176 extends from a suction port 236 in the cap to the suction cavity 220. Therefore, suction introduced by the suction producing device 32 can be transmitted to the suction cavity 220. As before, the porous member 222 permits the suction in the suction cavity 220 to permeate therethrough and be evenly distributed at the open end 232 of the suction cavity.

The circular flange 224 is sized and dimensioned such that when the cap 176 is lowered onto the base 172, the cap generally encloses the open upper end 186 of the base. In that regard, the circular flange 224 of the cap 176 is adapted to engage and seat with a shelf 238 on the base 172. To keep the cap 176 securely engaged with the base 172, one or more set screws 240 in the base can be manipulated.

The axial bore 226 passing through the cap 176 includes mating threads 242 that are formed closest to the lower end 216. The threads 242 are sized and dimensioned to permit about one tenth of a millimeter of axial travel per revolution of the trephine 174. Therefore, when the trephine 174 and the cap 176 are threadably engaged using threads 206, 242, the circular blade 208 will progress axially upwardly (as oriented in FIG. 10) about one tenth of a millimeter per revolution of the thumb wheel 202 of the trephine. As discussed above, either or both of the trephine 174 and the cap 176 can include indicia or other markings to provide a reference so that the eye surgeon can gauge axial movement corresponding to rotational movement.

Moving now to the recipient module 170, the fixator 178, as illustrated in FIG. 11, is similar to the threaded fixator 158 depicted in FIG. 9. Therefore, the fixator 178 generally includes all of the features and provides all of the advantages of fixator 158. Even so, the fixator 178 further includes a raised portion 244 of the body 246. The raised portion 244 includes a central bore 248 having threads 250. The threads 250 are sized and dimensioned to permit about one tenth of a millimeter of axial travel per revolution of the trephine 174. Therefore, when the trephine 174 and the fixator 178 are threadably engaged using threads 242, 250, the circular blade 208 will progress axially downwardly (as oriented in FIG. 10) about one tenth of a millimeter per revolution of the thumb wheel 202 of the trephine. As discussed above, either or both of the trephine 174 and the fixator 178 can include indicia or other markings to provide a reference so that the eye surgeon can gauge axial movement corresponding to rotational movement.

The recipient module 170 further includes a button suction adapter (BSA) 258. The BSA 258 is adapted to engage with a button suction adapter boss 260 on the fixator 178 and to be telescopically received with the axial bore 212 of the trephine 174. The BSA 258 includes a porous member 262 disposed within an annular suction cavity 264, an O-ring 266 encircling a distal end of a suction channel 268, an alignment flat 270, and an axial viewing channel 272.

In one embodiment, the pairs of threads 206, 242, 250 include a cooperating detent and follower, as well known in the art, such that discrete positions can be found, felt, and/or heard when rotating the threadably engaged components.

In operation, the PK unit 166 is utilized to aid the eye surgeon when performing the PK procedure. In that regard, the eye surgeon first employs the donor module 168. If the donor module 168 is not already assembled, the trephine 174 is lowered and telescopically received upon the stabilizer 188. Additionally, the cap 176 is lowered and received by the base 172 until the circular flange 224 engages the shelf 238 on the base and the open end 186 is closed. To secure the cap 176, the set screws 240 are tightened.

The eye surgeon next places a donor cornea (or entire eye) into the concave cavity 218. The donor cornea is oriented such that the epithelial side faces downwardly toward the trephine 174 housed in the base 172. With the donor cornea in the concave cavity 218, one or more of the suction producing devices such as suction producing device 32 (FIG. 1) is activated to provide suction to the suction cavities 192, 220. The suction passes through the porous members 194, 222 and releasably secures the donor cornea to the wall 230 within the concave cavity 218. Advantageously and as previously mentioned, the porous members 194, 222 prevent the cornea from being drawn into the suction cavity, prevent the cornea from being unnaturally distorted, prevent the cornea from shifting when incised, and the like. Also, not only does the suction on both sides of the circular blade 208 keep the cornea 48 of the eye 46 in a fixed position, the suction additionally keeps the cornea taught and/or tensioned on both sides of the circular blade to ensure a smooth, parallel cut surface.

With the donor cornea held in position, the eye surgeon raises the trephine 174 coaxially over the stabilizer 188 and into contact with the cap 176. When the trephine 174 and cap 176 are in close proximity, the eye surgeon begins to rotate the trephine 174 using the thumb wheel 202 (via the access cut-out 182) to threadably engage the trephine and the cap. Thereafter, the eye surgeon continues to rotate the trephine 174 using the thumb wheel 202 until the blade 208 encounters and begins to incise the donor cornea. As the eye surgeon further threadably advances the trephine 174 upwardly into the cap 176 using the thumb wheel 202, the blade 208 moves upwardly and eventually excises a “donor button” of corneal tissue from the donor cornea. The donor button has an outer wall that is parallel to the blade 208 as well as smooth.

After the donor button is cut away from the remainder of the cornea, the suction producing device 32 is deactivated, the suction is removed, and the donor button is very gently lifted from the concave cavity 218 with a forceps or other surgical instrument. Continuing, the set screws 240 are loosened, the cap 176 is removed from the base 172, and the trephine 174 is extracted.

Now that the donor button has been created and the trephine 174 removed from the base 172, the recipient module 170 is utilized. To begin, the fixator 178 and the BSA 258 are generally oriented and lowered until the trephine is upon the eye 46 of a patient, as described above, and until one portion of the BSA 258 engages the BSA boss 260 and another portion extends through the trephine 174 and engages the eye 46. When the first portion of the BSA 258 engages the BSA boss 260, the alignment flat 270 slides down over the O-ring 266 to promote a seal between the two pieces.

With the fixator 178 and the BSA 258 properly positioned, once again the suction producing device 32 is activated. When this occurs, suction is provided to the suction cavity 252. The suction permeates through the porous member 254 until reaching the open end 256 thereby releasably securing to the fixator 178 to the eye 46. Also, suction is communicated through the suction channel 268, to the suction cavity 264, and through the porous member 262 so that the eye is clamped. Therefore, the eye 46 is held by suction on both sides of the circular blade 208. This keeps the cornea 48 of the eye 46 in a fixed position as the incisions are made and keeps the cornea taught and/or tensioned on both sides of the circular blade 208 to ensure a smooth, parallel cut surface.

With the fixator 178 clamped to the eye 46, the same trephine 174 that was previously used within the donor module 168 to form the donor button is inverted and placed over the fixator 178 (as oriented in FIG. 10). The trephine 174 is then lowered and rotated into threaded engagement with the fixator 178 using the thumb wheel 202. The eye surgeon continues to rotate the trephine 174 using the thumb wheel 202 to drive the blade 208 downwardly into the fixator and toward the eye until the blade encounters and begins to incise the cornea of the patient. To view the incision and the inner portion of the eye, the eye surgeon can peer downwardly into the axial viewing channel 272. As the eye surgeon further threadably advances the trephine 174, the blade moves downwardly and eventually produces a circular cut entirely through the cornea of the patient and forms a patient button.

After the patient button has been formed, the suction producing device 32 is deactivated, the suction is removed, and the patient button is very gently lifted from the eye of the patient with a forceps or other surgical instrument. As such, the eye surgeon is generally able to remove the damaged, diseased, or undesirable central portion of corneal tissue from the eye. With the patient button removed and discarded, a central aperture or “bed” in the cornea of the eye is left behind. Since a circular blade 208 was employed to make the incision and form the bed, the outer wall of the central aperture is generally parallel to the downwardly driven blade and smoothly formed.

With the patient button removed and the bed exposed, the eye surgeon retrieves the donor button that was previously formed and places that donor button within the bed in the patients eye. This transfer of the donor button into the bed in the eye of the patient can again be performed with forceps or another surgical instrument. To complete the procedure, the eye surgeon fastens the donor button to the eye of the patient with a suture or stitching, reforms the anterior chamber with a sterile solution injected by a canula, and then tests the eye for a fluid tight seal using a dye.

Advantageously, since the same trephine 174 is used to excise the donor button and form the bed within the eye of the patient, the size and dimensions of the donor button very precisely match the size and dimensions of the bed in the eye of the patient. For example, the diameter of the donor button is similar to that of the bed, the outer wall of the button matches the outer wall of the central aperture, the angle of the outer wall on the donor button corresponds to the angle of the outer wall on the central aperture, and the like. However, different trephines may be used so long as the size, dimensions and angles of the circular blades are closely matched.

Additionally, since in the preferred embodiment the same trephine 174 is used to fashion the donor button and the bed, the loss of endothelial cells of the cornea is reduced, the surgeon is not required to add an undesirable amount of force to sutures or stitching to create a fluid impervious seal in the surgically repaired eye, any undesirable scarring (which can cause astigmatism) is reduced, and the eye of the patient is able to heal more quickly after the transplant, and the like.

While the PK unit 166 has been described as particularly beneficial in the performance of the PK procedure, those skilled in the art will recognize that the PK unit can also provide benefits to various other surgical procedures.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as ”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A surgical instrument releasably securable to an eye using suction, the surgical instrument comprising:
a body forming a suction cavity having an open end; and
a porous member disposed within the suction cavity, the porous member adapted to restrict absorption of the eye into the suction cavity through the open end and to permit the suction to permeate the suction cavity and reach the open end such that the surgical instrument is releasably securable to the eye using the suction.

2. The surgical instrument of claim 1, wherein the surgical instrument is formed from plastic.

3. The surgical instrument of claim 1, wherein the body is sized and dimensioned to have a diameter less than a diameter of a cornea portion of the eye.

4. The surgical instrument of claim 1, wherein the porous member is a microporous membrane.

5. The surgical instrument of claim 1, wherein the porous member is adapted to promote even distribution of the suction within the suction cavity proximate the open end.

6. The surgical instrument of claim 1, wherein one or more of the body and the suction cavity are annular.

7. The surgical instrument of claim 1, wherein the body includes a cylindrical bore, the cylindrical bore having an inner diameter less than a diameter of at least one of a vascular zone of an eye and a dilated pupil of the eye.

8. The surgical instrument of claim 7, wherein the cylindrical bore defines an inner body wall, the inner body wall including threads.

9. The surgical instrument of claim 1, wherein the body is semi-circular to form an open side.

10. The surgical instrument of claim 1, wherein the body has a curved lower surface adapted to correspond to a curvature of a corneal of the eye.

11. The surgical instrument of claim 1, wherein the body includes reference indicia on an upper exterior surface.

12. The surgical instrument of claim 1, wherein the body includes a quick connect mechanism adapted to couple with a suction generating device.

13. The surgical instrument of claim 1, wherein the body includes an integrally formed handle, the handle having a passage adapted to communicate the suction received by the suction cavity.

14. The surgical instrument of claim 1, wherein the body includes a handle having a quick connect mechanism.

15. The surgical instrument of claim 1, wherein the body includes a handle, the handle including at least one of a planar surface, a knurl, and a depression to promote gripping of the handle.

16. The surgical instrument of claim 1, further comprising a surgical apparatus for performing penetrating keratoplasty, the surgical apparatus including:
a base having a central stabilizer and an access cut-out, the central stabilizer disposed in the base;
a rotatable trephine adapted to be telescopically received upon the central stabilizer, the trephine including a rotation member, threads, and a circular blade, the rotation member accessible via the access cut-out; and
a cap adapted to releasably secure the trephine within the base and to releasably secure, along with the central stabilizer, a donor cornea, the cap including threads permitting the trephine to be threadably driven toward the donor cornea using the rotation member accessed through the access cut-out until a donor button is formed;
wherein the body of the surgical instrument includes threads permitting the trephine to be threadably driven toward the patient cornea using the rotation member until a patient button is formed after the trephine has been extracted from within the base, the patient button replaced by the donor button such that the penetrating keratoplasty is performed.

17. The surgical instrument of claim 16, wherein one or more of the central stabilizer and the cap employ suction to secure the donor cornea when forming the donor button.

18. The surgical instrument of claim 16, wherein the surgical apparatus employs suction to secure the donor cornea when forming the donor button.

19. The surgical instrument of claim 16, wherein the trephine is adapted to form the donor button in a first orientation and adapted to form the patient button in a second orientation, the second orientation inverted relative to the first orientation.

20. The surgical instrument of claim 16, wherein the rotation member is a thumb wheel.

21. The surgical instrument of claim 16, wherein the cap receives the donor cornea in a concave cavity formed in an upper surface of the cap.

22. A surgical instrument releasably securable to an eye using suction, the surgical instrument comprising:
a body forming an annular suction cavity, the annular suction cavity receiving the suction and having an open end in a concave lower surface, the open end directed toward the eye; and
a porous member disposed within the suction cavity, the porous member adapted to restrict absorption of the eye into the suction cavity through the open end and to permit the suction to permeate the suction cavity and reach the open end such that the surgical instrument is releasably securable to the eye using the suction.

23. The surgical instrument of claim 22, wherein the surgical instrument is disposable and releasably secured to a cornea portion of the eye.

24. The surgical instrument of claim 23 wherein the body includes reference indicia on an upper exterior surface and includes an integrally formed handle, the handle having a passage adapted to deliver the suction received by the suction cavity.

25. A surgical instrument releasably securable to an eye using suction, the surgical instrument comprising:
a body forming an annular suction cavity, the annular suction cavity receiving the suction and having an open end in a concave lower surface, the open end directed toward the eye;
a handle integrally formed with the body, the handle extending upwardly away from the eye and radially outwardly from the body, the handle including a passage adapted to deliver the suction to the suction cavity; and
a porous member disposed within the suction cavity, the porous member adapted to restrict absorption of the eye into the suction cavity through the open end and to permit the suction to permeate the suction cavity and reach the open end such that the surgical instrument is releasably securable to the eye using the suction.

26. The surgical instrument of claim 25, wherein the body and the handle are formed from a polymer.
